(19) United States
(12) Patent Application Publication
(10) Pub. No.: US 2007/0166413 A1
(43) Pub. Date: Jul. 19, 2007

(54) Cholestrol-reducing agent containing an n-3 fatty acid

(76) Inventors: Bernd Haber, Mainz (DE); Stephan Hausmanns, Wiesbaden (DE); Matthias Ruesing, Koeln (DE)

Correspondence Address: PROPAT, L.L.C., 425-C SOUTH SHARON AMITY ROAD, CHARLOTTE, NC, 28211-2841, US

(21) Appl. No.: 10/539,174

(22) PCT Filed: Dec. 22, 2003
(86) PCT No.: PCT/EP03/14713

§ 371 Date: Jun. 14, 2005

(30) Foreign Application Priority Data
Dec. 24, 2002 (DE) 102 61 067.3

(51) Int. Cl. A61K 31/22; A61K 36/48

(52) U.S. Cl. 424/757; 514/547

(57) ABSTRACT

This invention relates to a cholesterol-reducing active substance combination containing at least one cholesterol-reducing carob product, particularly water-insoluble carob fibers, at least one n-3 fatty acid and at least one cholesterol-reducing active substance. The invention also relates to a method for producing active substance combinations of this type and to the use thereof.

The invention relates to an active compound combination for reducing the cholesterol level containing at least one cholesterol-reducing carob product, in particular carob fibers, at least one n-3 fatty acid, and also at least one cholesterol-reducing active compound. The invention further relates to a method for producing such active compound combinations and also to their use.

In the context of an unbalanced diet, broad sections of the population display an elevated content of blood fat values, in particular blood cholesterol values. A cholesterol value of greater than 200 mg/dl, in particular LDL cholesterol values greater than 130 mg/dl, is considered one of the principal risk factors for cardiovascular disorders. Therefore, therapeutic treatment in the case of significantly increased cholesterol values, in particular LDL cholesterol, and increased blood fat values, is urgently necessary. To date, various approaches to a solution have been described for this. In addition to switching lifestyle and nutritional habits, which is of generally only slight efficacy, a number of special active compounds have been developed which intervene in different ways in the intake and metabolism of cholesterol. These are, inter alia, pharmacologically active substances such as statins (see, e.g., U.S. Pat. No. 4,231,938; U.S. Pat. No. 4,444,784; U.S. Pat. No. 4,346,227), inhibitors of bile acid resorption (see, e.g., U.S. Pat. No. 5,998,400; U.S. Pat. No. 6,277,831; U.S. Pat. No. 6,221,897) or bile acid sequestrants (see, e.g., U.S. Pat. No. 4,027,009). All these active compounds must be taken under medical supervision and monitoring.

The active compounds can also comprise cholesterol-reducing agents isolated from plant sources. Here, primarily, the cholesterol-reducing effect of a group of plant sterols, in particular phytosterols, phytostanols and the esters of said compound classes (see, e.g., WO-A-96/38047, WO-A-99/56558, U.S. Pat. No. 6,087,353) may be mentioned. Primarily the latter, however, are unsuitable for consumption by all population groups (e.g. exclusions for pregnant women or infants) and are frequently restricted in their application. Further natural cholesterol-reducing active compounds also include extracts from further plant sources, e.g. artichoke extracts, tocotrienol-rich extracts, garlic or gugulipid extracts.

In contrast, there are food components which have demonstrated repeatedly that, when consumed sufficiently, they can significantly reduce the risk of cardiovascular disorders, in particular also by reducing increased cholesterol levels. It is generally known that a high-fiber diet, compared with a low-fiber diet, is associated with a lower risk of cardiovascular disorders. In addition to whole-grain cereal (wheat, oats, barley, rye, but also cereal brans such as oat bran, rice bran, wheat bran, soy bran etc.) which is generally high-fiber, other fibers can also make a contribution to reducing cardiovascular risk and the increased cholesterol level. For instance, a number of water-soluble fibers, e.g. β-glucan (from oats or barley), psyllium, pectin or guar gum, demonstrate a reducing action on the blood cholesterol level (Brown et al. 1999; Am. J. Clin. Nutr. 69: 30-42).

In addition, water-insoluble carob fibers are known as food components, for example those produced by a method according to EP-A-0 616 780, which can significantly reduce serum cholesterol values, in particular LDL cholesterol (Zunft et al. 2001, Adv. In Ther. 18: 230-36). The HDL value remains constant here, so that the important LDL/HDL ratio is shifted toward the “good cholesterol”, and thus the cardiovascular risk decreases. The marked action of this insoluble, non-viscous preparation was the more surprising, since such reductions in cholesterol generally only occur in the case of viscous, soluble fibers.

Further food components which can contribute to a significant reduction in the risk of cardiovascular disorders comprise n-3 fatty acids. It is known that in most industrial countries the supply of n-3 fatty acids is deficient. In contrast, in particular the total fat content in the diet and the supply of saturated fatty acids and n-6 fatty acids is too high. This is based on a change in our food composition which has taken place primarily in the last approximately 150 years and which is correlated with the occurrence of various chronic disorders (of civilization), in particular cardiovascular disorders, the principal cause of death in industrial countries. A multiplicity of studies has found in the interim that the targeted increase in the supply of n-3 fatty acids, in particular all-cis-5,8,11,14,17-eicosapentaenoic acid (EPA) and all-cis-4,7,10,13,16,19-docosahexaenoic acid (DHA) can significantly reduce the cardiovascular risk [GISSI-Prevenzione Investigators (Gruppo Italiano per lo Studio della Sopravvivenza nell'Infarto miocardico), Dietary supplementation with n-3 polyunsaturated fatty acids and vitamin E after myocardial infarction: results of the GISSI-pevenzione trial. Lancet. 1999; 354: 447-455; Burr et al. Effects of changes in fat, fish and fibre intake on death and myocardial reinfarction: diet and reinfarction trial (DART), The Lancet, 1989, 757-761). Correspondingly, many different organizations (WHO, FAO, AHA, ISSFAL, British Nutrition Foundation and many others) recommend significantly increasing the supply of n-3 fatty acids. Here (depending on recommendation), a deficiency of supply of at least 0.5 to 1.5 g of n-3 fatty acids is found. Most recommendations relate here to the supply of n-3 fatty acids (in particular DHA and EPA) by regular consumption (at least 2×weekly) of fatty sea fish. Although the beneficial effects on reduction of cardiovascular risk by n-3 fatty acids are not clear in detail, they are primarily associated with beneficial effects on some of the main risk factors for cardiovascular disorders such as arteriosclerosis, high blood pressure, plasma triglyceride level, arrhythmias and heart frequency variability. Interestingly, the n-3 fatty acids appear to have no effect, or only a slight effect, on a further main risk factor, the cholesterol level. At best, a slight shift in the LDL/HDL ratio toward the “good cholesterol” is discussed (Gylling and Miettinen, Curr Control Trials Cardiovasc Med 2001, 123-128).

However, the effects which can be achieved with all these food components are significantly below those which are achieved with therapeutic active compounds, and are thus far lower than desirable. Even if a diet enriched with fibers, in particular carob fibers, can make a contribution toward controlling the cholesterol level and the blood fat values, in many cases, in particular in the case of very high cholesterol levels (total cholesterol >300 mg/dl) it is insufficient for a lasting reduction. Likewise, a diet enriched with n-3 fatty acids, in particular with all-cis-9,12,15-octadecatrienoic acid (ALA), EPA and DHA, can make a valuable contribution to generally reducing cardiovascular risk and to improving general health, but in many cases, in particular in the case of an increased cardiovascular risk (e.g. after a heart attack), this alone is not sufficient.

A cholesterol-reducing interaction between carob products, n-3 fatty acids and cholesterol-reducing active compounds is not known. However, there are indications that viscous fibers such as pectin, can have, together with n-3 fatty acids, a synergistic effect in cholesterol reduction (V. Bartz 2002, Ernährung & Medizin 17, 149-150). Since carob products, in particular carob fibers, are not viscous, a cholesterol-reducing interaction is not obvious, certainly not a synergistic action. For instance, even an antagonistic action of water-insoluble fibers of carob fruit flesh with the viscous fiber carob bean meal has been described (Peres-Olleros et al. 1999; J. Sci. Food Agric. 79, 173-178). Also, for example, additional daily administration of 56 g of oat bran to a therapy with nicotinic acid showed no further reduction in LDL cholesterol (Keenan J. M. et al.: J. Fam. Pract., 34 (1992), 313-319).

The purely pharmacological cholesterol-reducing agents have the disadvantage that, to achieve the therapeutic aims, sometimes considerable concentrations have to be used. Here, unwanted, sometimes life-threatening side effects can occur, also in combination with other therapeutic agents. Combination therapies for increasing the activity with various cholesterol-reducing active compounds or else other therapeutic agents, e.g. for cardiovascular disorders, cannot always be used because of various hazardous contraindications. For instance, combinations of fibrates with statins demonstrate an increased risk of myopathy syndromes which in the case of combinations of cerivastatin with gemfibrozil, can even end fatally.

Furthermore, saturation effects are known which mean that, with an increased intake of the active compound, only slight additional reductions of the cholesterol level are achieved. A further disadvantage is the high costs which can occur in the case of long-term therapies using the generally very expensive pharmacological cholesterol-reducing agents.

In the case of the cholesterol-reducing agents isolated from plant sources (e.g. phytosterols), there are quantity limitations to avoid unwanted side effects.

Therefore, there is still a requirement for cholesterol-reducing agents which, with the same, or even improved activity, reduce the amounts of the respective active compound administered and thus decrease the possible side effects and costs, in particular of long-term therapies.

This object is achieved by providing an active compound combination for reducing the cholesterol level comprising at least one carob product, in particular carob fibers, having a high content of fibers, at least one n-3 fatty acid, and also at least one cholesterol-reducing active compound. In this case, when the inventive active compound combination is applied, in addition to the above-described effect of total cholesterol reduction, a shift in the ratio of HDL and LDL toward the “good” HDL cholesterol occurs.

Furthermore, this synergistic reduction of the cholesterol level by the inventive active compound combination is advantageously supplemented by the known beneficial effect of n-3 fatty acids on the cardiovascular system (see above).

Independently of the above-described beneficial effects on cardiovascular health, the inventive dietetic foods achieve an additional beneficial effect on health by means of an increased supply of n-3 fatty acids. DHA, which is preferably used according to the invention, plays a particular role here. In addition, the inventive active compound combination can compensate for a depletion of the body in essential n-3 fatty acids which, experience shows, can result after administration of dietary fibers, and in particular as an unwanted side reaction in drug treatment of high cholesterol values with statins.

A therapy using the inventive active compound combination, therefore, promotes health in general and not just actual cardiovascular health.

Carob products in the context of the invention are the carob fruit itself and also components obtained therefrom. Those which are preferably used in the context of the invention are carob fibers which are characterized by a high content of total dietary fibers, determined by AOAC method 985.29, of at least 30% by weight, preferably at least 60% by weight, particularly preferably at least 80% by weight (in each case based on the dry mass). Their content of water-insoluble dietary fibers determined by AOAC method 991.42 is at least 25% by weight, preferably at least 50% by weight, particularly preferably at least 70% by weight.

To produce the carob fiber product, in particular removal of the water-soluble carob components from the fruit flesh freed from the carob seeds and heating for the (partial) denaturation of the condensed tannins are necessary. Further process steps comprise washing and separation steps, drying, grinding and if appropriate sifting. This produces fiber lengths of <250 μm, preferably <150 μm, in particular <100 μm. Particular preference is given to the methods as claimed in EP-A-0 616 780 and according to the unpublished PCT/EP03/08636. The preparations thus produced exhibit a pronounced hypocholesterolemic action and moderate triglyceride-reducing action and can be used to enrich foods.

For the purposes of the invention, n-3 fatty acids (omega-3 fatty acids, ω-3 fatty acids) are taken to mean long-chain polyunsaturated fatty acids (PUFAs) having a chain length >C12 having at least two double bonds, the first of the at least two or more double bonds, starting from the alkyl end, being constituted between the carbon atoms C3 and C4 (see table 1). Here, the n-3 fatty acids can be both present as free fatty acids, esters, triglycerides, phospholipids, glycolipids, sphingolipids, waxes or sterol esters, or can have been enriched in the form of their monohydric alcohol esters by chemical or biocatalytic transesterification of the triglycerides, e.g. using suitable enzymes (lipases). All of these substances and also products which comprise these substances at concentrations of at least 15 area-% of TFA (see below) are summarized hereinafter by the terms n-3 fatty acid or n-3 active compounds; these terms are used synonymously.

TABLE 1
n-3 fatty acids

| | | Trivial name, |
|---|---|---|
| | IUPAC name | abbreviation |
| C18:3 | all-cis-9,12,15-Octadeca- | α-Linolenic acid ALA |
| | trienoic acid | |
| C18:4 | all-cis-6,9,12,15-Octadeca- | Stearidonic acid |
| | tetraenoic acid | |
| C20:3 | all-cis-11,14,17-Eicosatrienoic acid | |
| C20:4 | all-cis-8,11,14,17-Eicosa- | ETA |
| | tetraenoic acid | |
| C20:5 | all-cis-5,8,11,14,17-Eicosa- | EPA, timnodonic acid |
| | pentaenoic acid | |
| C22:3 | all-cis-13,16,19-Docosatrienoic acid | |
| C22:5 | all-cis-7,10,13,16,19-Docosa- | DPA fish oil w-3 |
| | pentaenoia acid | |
| C22:5 | all-cis-4,7,10,13,16-Docosa- | DPA Protists w-6 |
| | pentaenoic acid | |
| C22:6 | all-cis-4,7,10,13,16,19- | DHA |
| | Docosahexaenoic acid | |

Preference for the purpose of the invention is given to the use of an n-3 active compound having a content of n-3 fatty acids of at least 20 area-% of TFA (area-% relates to the AOCS official method Ce 1b-89; TFA=total fatty acid). Particular preference is given to a content of at least 30 area-% of TFA, in particular of at least 40 area-% of TFA and very particularly preferably of at least 60 area-% of TFA.

Further preference for the purposes of the invention is given to mixtures of the various n-3 active compounds, preferably of at least 2 of the n-3 active compounds DHA, EPA and ALA, and particularly preferably a mixture of the n-3 active compounds DHA and EPA. Very particular preference is given to the use of EPA or DHA as main constituent of the n-3 active compound, in particular the use of DHA as single n-3 active compound.

A suitable source of an abovementioned mixture of EPA and DHA is fish oils. A suitable source of ALA is plant oils, in particular linseed oil or hemp oil inter alia.

Particular preference is given to n-3 active compounds which are isolated from microorganisms. Preferred microorganisms are organisms of the Stramenopiles (or Labyrinthulomycota), particularly preferably of the order Thraustochytriales, (Thraustchytriidea), in particular of the genera Schizochytrium, Thraustochytrium and Ulkenia, and also Dinoflagellates (Dinophyta), preferably Cryplthecodinium, in particular C. cohnii, which are preferably suitable for producing DHA at a concentration of at least 20 area-% of TFA, preferably at least 30 area-% of TFA, and particularly preferably at least 40 area-% of TFA DHA. In this case, with respect to the production of n-3 fatty acids, the following publications are incorporated in particular by reference: WO-A-91/07498, WO-A-91/11918, WO-A-96/33263 and WO-A-98/03671.

Further suitable sources of EPA and/or DHA are also, e.g., microalgae such as Euglena (JP-A-60-196157), Nannochloropsis, Phaeodactylum and others (Tonon et al., Long chain polyunsaturated fatty acid production and partitioning to triacylglycerols in four microalgae. Phytochemistry 2002, 15-24), but also bacteria, preferably e.g. Shewanella, Vibrio or Moritella (Cho and Mo, Screening and characterization of eicosapentaenoic acid-producing marine bacteria, Biotechnology Letters 1999, 215-218; JP-A-2000/245442; JP-A-63-216490, JP-A-2001/309797).

Further possible sources of n-3 fatty acids are transgenic organisms, preferably microorganisms and plants.

In addition, use can be made for the purposes of the invention of n-3 active compounds which are purified or concentrated by various methods known to those skilled in the art (e.g. chromatography, absorption or adsorption methods, winterization etc.) from oils as described above (e.g. fish oils, vegetable oils or oils from microorganisms).

For the purposes of the invention, cholesterol-reducing active compounds are taken to mean active compounds which can reduce an elevated cholesterol level (>200 mg/dl), in particular LDL-cholesterol level >130 mg/dl. These are distinguished in that they affect specifically defined metabolic processes and as a result secondarily lead to a reduction of the LDL cholesterol and the total cholesterol (generally between 10-55%).

For the purposes of the invention, the active compounds comprise cholesterol-reducing substances from the group of statins, bile acid resorption inhibitors, and bile acid sequestrants, cholesterol absorption inhibitors, fibrates, nicotinic acid derivatives, but also the group of phytosterols and plant stanols, and also cholesterol-reducing plant extracts.

The active group statins is taken to mean compounds such as lovastatin [see FIG. 1 below] (e.g. U.S. Pat. No. 4,231,938), pravastatin (e.g. U.S. Pat. No. 4,346,227), simvastatin [see FIG. 2 below] (e.g. U.S. Pat. No. 4,444,784), fluvastatin (e.g. U.S. Pat. No. 5,354,772), atorvastatin (e.g. U.S. Pat. No. 5,273,995) or cerivastatin (e.g. U.S. Pat. No. 5,177,080) which act specifically in the liver via an inhibition of cholesterol synthesis (HMG CoA reductase inhibitors). These active substances have been described many times and are widely used for cholesterol reduction as medicament and for therapy (e.g. U.S. Pat. No. 6,180,660)

[image]

For the purposes of the invention, inhibitors of bile acid resorption are taken to mean substances which inhibit the reuptake of bile acids in the stomach/ileum via a receptor-mediated process. These are, in particular, benzothiazepine derivatives (e.g. U.S. Pat. No. 5,998,400, U.S. Pat. No. 6,277,831), benzothiepine-1,1-dioxide derivatives (e.g. U.S. Pat. No. 6,221,897, WO-A-97/33882), in particular compounds according to FIGS. 3 and 4 below which cause a blockade of bile acid resorption specifically in the stomach, in particular in the ileum.

[image]

(where R═C₆H₄NHZR₃; R¹, R⁴, R⁵=Me, Et, Pr, Bu; R²═H, OH, NH₂, amino(alkyl); R³=sugar radical; Z=—(C═O)ₙ-(C₀ -C₁₆)-alkyl-, (C═O)ₙ-(C₀-C₁₆)-alkyl-NH—, —(C═O)ₙ -(C₀ -C₁₆)-alkyl-O—, —(C═O)ₙ-(C₀-C₁₆)-alkyl-(C═O)ₘ or a covalent bond; n=0 or 1; m=0 or 1, and also their salts)

[image]

(where R¹=Me, Et, Pr, Bu; R²═H, OH; R³=sugar radical; Z=—(C═O)ₙ-(C₀-C₁₆)-alkyl-, -(C═O)ₙ—(C₀-C₁₆)-alkyl-NH—, —(C═O)ₙ-(C₀-C₁₆)-alkyl-O—, —(C═O)ₙ-(C₀-C₁₆)-alkyl-(C═O)ₘ or a covalent bond; n=0 or 1; m=0 or 1, and also their salts)

Bile acid sequestrants act as polymeric ion exchange resins in the stomach specifically on bile acids, but also cholesterol, and lead to an intensified excretion of said substances. This group of active compounds comprises, inter alia, cholestyramine, colestipol or colesevlam hydrochloride. The two said compounds are distinguished by a markedly lower activity than statins or inhibitors of bile acid resorption.

Cholesterol absorption inhibitors are active compounds which in the stomach inhibit the receptor-mediated transport of cholesterol and thus increase the excretion of cholesterol which ultimately leads to a moderate reduction of the serum cholesterol level. These include, in particular, hydroxy-substituted azetidinone cholesterol absorption inhibitors of the group 1-(4-fluorophenyl)-3(R)-[3(S)-(4-fluorophenyl)-3-hydroxypropyl)]-4(S)-4-hydroxyphenyl)-2-azetidinone) and 1-(4-fluorophenyl)-3(R)-[3(R)-(4-fluorophenyl)-3-hydroxypropyl)]-4(S)-4-hydroxyphenyl)-2-azetidinone) and their pharmacologically active salts or else substituted β-lactam cholesterol absorption inhibitors (e.g. WO-A-95/35277, WO-A-02/058733, WO-A-02/50060).

The group of the fibrates comprises, inter alia, clofibrate, etophyllin clofibrate, bezafibrate, ciprofibrate, clinofibrate, binifibrate, lifibrol, fenofibrate, gemfibrozil or etofibrate. Depending on the disease profile, fibrates have a moderately reducing action on LDL cholesterol with a slight improvement of the HDL cholesterol values. Serum triglycerides are more strongly affected by fibrates.

For the purposes of the invention, nicotinic acid derivatives are natural or synthetically produced nicotinic acid, its esters or synthetic derivatives, e.g. niceritrol, nicofuranose, β-pyridylcarbinol or acipimox. This group of substances has a moderate effect on total and LDL cholesterol with simultaneously improved HDL cholesterol levels.

For the purposes of the invention, phytosterols are taken to mean 4-desmethylsterols, 4-monomethylsterols and 4,4-dimethylsterols and the respective esters and also plant extracts, mixtures and foods rich in phytosterols. These comprise β-sitosterol, campesterol, stigmatosterol, brassicasterol, desmosterol, chalinosterol, poriferasterol and clinosterol and all their natural or synthetic or isomeric derivatives. Plant stanols are taken to mean hydrogenated plant sterols, e.g. campestanol, sitostanol and the respective esters and also plant extracts, mixtures and foods rich in plant stanols.

Further plant extracts having cholesterol-reducing activity comprise, inter alia, artichoke extracts and extracts of garlic and gugulipid. They have long been used as natural therapeutic agents and exhibit moderate activity on the total and LDL cholesterol level.

The inventive agents comprise a carob product, in particular carob fibers, at least one cholesterol-reducing active compound and at least one n-3 fatty acid. In addition, the cholesterol-reducing agents can comprise customary additives such as solvents, fillers, carriers such as methylcellulose, sweetening carbohydrates and other sweeteners, flavorings, antioxidants and preservatives. The combination of a carob product, in particular carob fibers, with at least one n-3 fatty acid and at least one active compound can also be administered in the form of two or more different administration forms. Suitable applications for the carob products, in particular the carob fibers, and for the n-3 fatty acids are current food applications such as bakery products, cereals, snack bars or fruit bars or drinks powders. Furthermore, direct addition of the carob product, in particular the carob fibers, and of the n-3 fatty acids to self-produced foods and also use in food supplement-type form (inter alia tablets, dragees, hard or soft capsules, sachets, granules, bars, etc.) is also possible, while the active compounds are rather administered in a manner typical of drugs (inter alia tablets, dragees, hard or soft capsules, sachets, granules etc.).

The inventive dietetic foods comprise the food components in amounts which are required to achieve a therapeutic effect in 2- to 4-times daily administration.

The carob product or the carob fiber component is present in the inventive products at concentrations which cause a marked cholesterol reduction or affect the HDL/LDL ratio in a beneficial manner. The daily dose of carob fiber can be in the range 1-25 g, customarily 5-15 g.

The n-3 fatty acids are present in the inventive products at concentrations which, in synergy with the above-described carob products, cause a marked cholesterol reduction and affect the HDL/LDL ratio in a beneficial manner. The daily dose of n-3 fatty acids can be in the range from 50 mg to 10 g, customarily 100 mg to 5 g, and preferably 200 mg to 2 g.

Carob product, in particular carob fibers, and n-3 fatty acids, are used in these amounts in combination with the usual daily doses of the active compounds when a particularly extensive reduction of the cholesterol level is sought. For the active compound concentrations previously necessary for individual application, the usage concentrations can be reduced, owing to synergies, by up to 50-90%. The additives possibly present can be added at concentrations expediently of 1-90% by weight, in particular of 10-60% by weight, based on the respective preparation form.

The inventive active compound combination can be taken at one defined daily timepoint, or distributed over the day, the weight ratios of active compound, carob product, in particular carob fibers, and n-3 fatty acid in the intake of relatively small doses corresponding to the abovementioned ratios.

To produce the inventive agents, preferably a process can be followed such that the desired amounts of carob product, in particular carob fibers, n-3 fatty acids and active compound are mixed with one another, spray dried, freed from the solvent, agglomerated and/or instantized. Furthermore, all customary methods of food technology or else gallenical production methods such as pressing, kneading or dragee-coating can be used. The n-3 fatty acids can be added to the mixture in pure form, or encapsulated or microencapsulated, all methods familiar to those skilled in the art such as coacervation, spray drying or fluidized-bed drying being able to be used for the encapsulation or microencapsulation. Inclusion in liposomes or micelles is also possible.

In addition, the n-3 fatty acids can be added to the mixture in a form which permits a continuous release (slow release) of the fatty acids in the body. Suitable methods for producing these “slow release” formulations are, for example, coating methods, or the use of suitable capsule matrices in (micro)encapsulation.

Furthermore, the carob product itself, in particular the carob fibers, can be used as carrier or matrix for the n-3 fatty acids.

In the case of joint administration according to the present invention, it has been found that the combined intake of carob products, in particular carob fibers, n-3 fatty acids and cholesterol-reducing active compounds leads to a markedly stronger reduction of the cholesterol level than the sum of the effects in the case of administration of the individual components. It is surprising in this case that the additional administration of carob products, in particular carob fibers, and n-3 fatty acids to the active compounds do not reduce the activity of the active compounds by non-specific interference, but that the effects observed go markedly beyond the effects achievable in the case of individual administration of the three groups of substances.

The inventive agents thus permit a greater reduction of the cholesterol level to be achieved than hitherto, which is frequently also therapeutically desirable, or else achieve effects at a comparable level as previously, but using lower amounts of active compound. In particular, unwanted side effects which frequently occur in the administration of cholesterol-reducing active compounds can thus be reduced or avoided entirely. The inventive active compound combination is thus an important advance in drug therapy of hypercholesterolemia or hyperlipidemia.

The inventive active compound combinations are expediently used in a suitable preparation which is matched to the optimally-acting ratios. For this, e.g. pulverulent or tablet-form preparations for dissolution, but also chewing tablets, come into consideration. These preparations can also comprise further constituents (additives) for improving the dissolution, such as soluble carriers, tablet disintegrants, for example starch, cellulose, bentonite, pectin or peroxides and carbonates in combination with organic acids and generally colorants, sweeteners such as sucrose, glucose, fructose and other carbohydrates, sugar alcohols, e.g. sorbitol, xylitol, maltitol and isomalt, or non-nutritive sweeteners, e.g. acesulfame-K, cyclamate, saccharin, sucralose or aspartame, and in particular flavorings for improving acceptance.

The inventive agents may also be administered, however, separately in the form of a drug preparation of the active compound and in the form of a food or food supplement comprising the carob product, in particular carob fibers, and the n-3 fatty acids. In particular, in this case, use can be made of the carob product, in particular the carob fibers, as carrier of the n-3 fatty acids. In addition, separate administration of two foods or food supplements is possible, one food or food supplement comprising the carob product and the other comprising the n-3 fatty acid(s). For the active compound, the customary drug administration forms such as tablets, capsules, solution for consumption as drops or pulverulent preparation or granules to be dissolved come into consideration. In the case of this combination therapy, a suitable food is in principle any food into which the carob product and the n-3 fatty acid can be incorporated, limits resulting from the properties of the food component and also from the intended field of application. Particularly suitable foods would therefore be those on a cereal basis, such as bakery products, cereals, snack and fruit bars, desserts, special diet preparations such as drinks, and in particular powdered drinks based on milk, fruit concentrates or fruit powders, carbohydrates or sugar alcohols. In the case of phytosterols and plant stanols, in addition, fatty foods come into consideration, e.g. vegetable spreading fats, dressings and milk products.

The invention will be described hereinafter on the basis of an example.

### EXAMPLE

Pulverulent Preparation (for One Portion Size)

Lovastatin (MSD Sharp and Dome GmbH, 10 mg D-85540 Haar)

Carob fibers (Caromax®, Nutrinova, Frankfurt) 3 g

DHA-rich algal oil (DHA content 43 area-% TFA; 150 mg Nutrinova, Frankfurt)

Xanthan (stabilizer) 150 mg

Vanillin 15 mg

Suspend the preparation in 150 ml of tepid milk by stirring and drink.

1. A cholesterol-reducing agent comprising from 1 to 15 g of at least one carob product, n-3 fatty acid comprising at least DHA and at least one cholesterol-reducing active compound, the cholesterol-reducing active compound being selected from one or more of the following substances: statins, inhibitors of bile acid resorption, cholesterol absorption inhibitors, fibrates, nicotinic acid derivatives, bile acid sequestrants, phytosterols, plant stanols and also cholesterol-reducing plant extracts, said agent providing a greater reduction in cholesterol level than the sum of the effects when the carob product, n-3 fatty acid or cholesterol-reducing active compound arc administered alone,
said agent further providing a reduction of total cholesterol of at least 10% when said n-3 fatty acid consists of DHA alone.

2. The agent as claimed in claim 1, wherein the carob product is carob fruit flesh or a product produced from carob frit flesh.

3. The agent as claimed in claim 1, wherein the carob product is carob fiber.

4. The agent as claimed in claim 3, wherein the carob fiber is insoluble in water.

5. The agent as claimed in claim 1, wherein the n-3 fatty acid is a single polyunsaturated fatty acid having a chain length >C12 having at least two double bonds, or its ester, triglyceride, phospholipid, glycolipid, sphingolipid, wax or sterol ester.

6. The agent as claimed in claim 5, wherein the first of the at least two double bonds starting from the alkyl end is constituted between the carbon atoms C3 and C4.

7. The agent as claimed in claim 5, wherein the n-3 fatty acid is selected from one or more of the following substances: all-cis-9,12,15-octadecatrienoic acid (ALA), all-cis-6,9,12,15-octadecatetraenoic acid, all-cis-11,14,17-eicosatrienoic acid, all-cis-8,11,14,17-eicosatetraenoic acid, all-cis-5,8,11,14,17-eicosapentaenoic acid (EPA), all-cis-13,16,19-docosatrienoic acid, all-cis-7,10,13,16,19-docosapentaenoic acid (DPA) and all-cis-4,7,10,13,16,19-docosahexaenoic acid (DHA).

8. The agent as claimed in claim 7, wherein the n-3 fatty acid is all-cis-4,7,10,13,16,19-docosahexaenoic acid (DHA).

9. The agent as claimed in claim 5, wherein the n-3 fatty acid is not derivatized.

10. A cholesterol-reducing combination preparation comprising from 1 to 15 g of at least one carob product, n-3 fatty acid comprising at least DHA and at least one cholesterol-reducing active compound in separate administration forms, said cholesterol-reducing combination preparation providing a reduction in total cholesterol of at least 10% when said n-3 fatty acid consists of DHA alone.

11. The cholesterol-reducing combination preparation as claimed in claim 10, in which the carob product and the n-3 fatty acid are present in a food.

12. The cholesterol-reducing combination preparation as claimed in claim 10, in which the active compound is present in a food or a drug.

13. A method for producing an agent as claimed in claim 1, which comprises mixing at least one carob product and at least one n-3 fatty acid and at least one cholesterol-reducing active compound with one another.

14. A drug comprising an agent as claimed in claim 1.

15. A drug as claimed in claim 11 wherein said drug is a cholesterol-reducing drug.

16. A drug as claimed in claim 11 wherein said drug is a hypereholesterolermia, hyperlipidemia or arteriosclerosis.

17. A drug as claimed in claim 11, said drug positively shifting the HDL/LDL ratio.

18. An agent as claimed in claim 1 said agent further comprising an additional supply of n-3 fatty acids.

19. Pet food comprising an agent as claimed in claim 1.

20. (canceled)

21. An agent as claimed in claim 1, wherein said n-3 fatty acid is derived from vegetable oil or oils from microorganisms.

22. A cholesterol-reducing agent comprising at least one carob product, at least one n-3 fatty acid and at least one cholesterol-reducing active compound,
said n-3 fatty acid consisting of one or more of: all-cis-9,12,15-octadecatrienoic acid (ALA), all-cis-6,9,12,15 -octadecatetraenoic acid, all-cis-11,14,17-eicosatrienoic acid, all-cis-13,16,19-docosatrienoic acid, all-cis-7,10,13,16,19-docosapentaenoic acid (DPA) and all-cis-4,7,10,13,16, 19-docosahexaenoic acid (DHA),
said cholesterol-reducing active compound being selected from one or more of the following substances: statins, inhibitors of bile acid resorption, cholesterol absorption inhibitors, fibrates, nicotinic acid derivatives, bile acid sequestrants, phytosterols, plant stanols and also cholesterol-reducing plant extracts,
wherein said agent provides a greater reduction in cholesterol level than the sum of the effects when the carob product, n-3 fatty acid or cholesterol-reducing active compound are administered alone.

23. An agent as claimed in claim 1, wherein said n-3 fatty acid is administered in a daily dose ranging from 50 mg to 600 mg.

24. An agent as claimed in claim 1, wherein said cholesterol-reducing compound is present at 10 to 50% of the dosage which would be recommended in the absence of said carob product and said n-3 fatty acid.
